(19) United States
(12) Patent Application Publication
(10) Pub. No.: US 2002/0022779 A1
(43) Pub. Date: Feb. 21, 2002

(54) Apparatus and methods for diagnosing osteoporosis and other diseases with MR imaging

(76) Inventor: Katsumi Kose, Kashiwa-shi (JP)

Correspondence Address: SHLESINGER, ARKWRIGHT & GARVEY LLP, 3000 South Eads Street, Arlington, VA, 22202, US

(21) Appl. No.: 09/916,446

(22) Filed: Jul. 30, 2001

(30) Foreign Application Priority Data
Aug. 8, 2000 (JP) 2000-275673

(51) Int. Cl. A61B 5/055

(52) U.S. Cl. 600/422; 600/415

(57) ABSTRACT

A compact magnet and an RF probe which can accommodate a human extremity such as a heel are used to construct a compact MRI system for diagnosis and follow-up of osteoporosis and other diseases. Methods for measuring and calculating proton density in inhomogeneous static magnetic field, magnetic field gradients, and RF magnetic field are provided using 2D spin-echo image acquisitions with external reference materials and image analyses. The measured proton density of bone marrow is used for computation of trabecular bone volume fraction, which can be used for diagnosis of osteoporosis and other diseases.

### FIELD OF THE INVENTION

This invention generally relates to magnetic resonance imaging (MRI) utilizing nuclear magnetic resonance (NMR) phenomena. It more particularly relates to a specialized MRI system used for diagnosis of osteoporosis and other diseases.

### BACKGROUND OF THE INVENTION

Osteoporosis is a widespread disease characterized by bone loss usually associated with aging. It is a complex and chronic disease that may not be recognized until resulting fractures late in life. In many countries around 50% of women above age of 70 is osteoporosis and to overcome it has become an important social requirement. For diagnosis and follow-up of osteoporosis, measurement of bone mineral density (BMD) is indispensable. The conventional methods for the BMD measurement are based on X-ray attenuation measurements through a bone. Among them, DEXA (Dual-Energy X-ray Absorptiometry) is now the standard method, but cannot be used repeatedly nor for young women because of the ionizing radiation. Thus the X-ray BMD measurement should be replaced by some noninvasive and high precision method such as bone marrow volume measurement using MRI as described below, because MRI has no ionizing radiation and spatial localization capability is guaranteed.

The bones of a human skeletal system have a dense outer shell made of cortical bone (alternatively termed “compact” or “dense” bone). Inside the cortical bone, many regions have a mesh or network of trabecular bone (alternatively termed “spongy” or “cancellous” bone), made up of roughly the same material as the cortical bone. The regions enclosed by the cortical bone and/or trabecular network are filled with “bone marrow”, of which major components are water and lipid. Since the protons (hydrogen nuclei) of the water and lipid can be measured using MRI, the bone marrow volume can be quantified using MR images. Thus quantitative bone MRI can be a complementary method for the measurement of BMD because bone is almost chemically homogeneous. Studies according to this concept are found in the following references: Jara et al., “High-Resolution Variable Flip Angle 3D MR Imaging of Trabecular Microstructure in Vivo”, Magnetic Resonance in Medicine 29: 528-539, 1993; Wehrli et al., “Cancellous Bone Volume and Structure in the Forearm: Noninvasive Assessment with MR Microimaging and Image Processing”, Radiology 206: 347357, 1998; L. Hilaire et al., “High-Speed Spectroscopic Imaging for Cancellous Bone Marrow R₂* mapping and Lipid Quantification”, Magnetic Resonance Imaging, 18: 777-786, 2000.

MRI is by now a commercially available and widely accepted non-invasive method for measuring information about internal structures of living systems and heterogeneous materials. However, most of medical MRI are developed for a whole human body and require a large cost and space. For the measurement of bone marrow volume, a compact MRI system for a part of a human body is appropriate because bone density for a whole human body can be well estimated from a measurement of a part of bone.

### SUMMARY OF THE INVENTION

The most serious fracture for osteoporosis patients is the hip fracture because the patients cannot move by themselves for more than several months. To acquire MR images of a hip bone (femur) for diagnosis of osteoporosis, a whole body MRI system is indispensable, requiring a large cost for the patients. However, because correlations among densities of bones at various parts of a human body are very high, the bone density at the femur can be well estimated from those of other bones.

In this invention, calcaneus is used for the bone density measurement using MRI because of the following reasons: first, calcaneus is made of homogeneous trabecular bone and reflects the whole-body bone status very well; second, calcaneus is a peripheral part of a human body and easy to measure using a compact MRI system; third, the magnet, usually the heaviest unit of an MRI system, can be placed in the lower level and the MRI system becomes mechanically stable. For the measurement of a heel, an RF probe box having an oval aperture with a support pad to fix the heel in the RF coil is used. In the RF coil, external reference materials are placed and imaged together with the heel for NMR signal intensity calibration.

Quantification of materials using a compact MRI system gives rise to several new problems, because the magnet, gradient coil, and RF coil are small compared to those of a whole body MRI and the inhomogeneity of the static magnetic field, magnetic field gradients, and RF magnetic field become large compared to that of a whole body MRI. To measure proton density of bone marrow of calcaneus in such inhomogeneous magnetic fields, spin-echo imaging sequences with different echo times are used to image a heel together with external reference materials. By combining measurements with different echo times and reference materials, T₂ decay effects and spatially varying image intensities are corrected. As a result, the proton density of bone marrow is obtained and used for evaluation and follow-up of osteoporosis and other diseases.

### BRIEF DESCRIPTIONS OF THE DRAWINGS

FIG. 1 is a schematic overview of a compact MRI system for imaging of a heel;

FIG. 2 is a schematic representation of an RF probe box with an oval aperture used for imaging of a heel;

FIG. 3 schematically depicts a solenoid coil and matching circuit for the RF probe;

FIG. 4 is a schematic cross-sectional depiction of a support pad for a heel and external reference materials, inserted in the open-bore of the RF probe shown in FIG. 2;

FIG. 5 is a schematic cross-sectional depiction of another type of the support pad for a heel and external reference material, inserted in the open-bore of the RF probe shown in FIG. 2;

FIG. 6 is a standard 2D spin-echo pulse sequence used to quantify the proton density of bone marrow in calcaneus;

FIG. 7 is a flow diagram of one method for calculating proton density using the MRI system shown in FIG. 1;

FIG. 8 schematically shows a vertical cross-sectional image of a heel with external reference phantoms;

FIG. 9 schematically shows a vertical cross-sectional image of a water phantom for intensity correction of images of heels with external reference phantoms;

FIG. 10 is a semilogarithmic plot of spin-echo image intensities of a plant oil phantom plotted against the echo time;

FIG. 11 is a large flip-angle excitation 2D spin-echo pulse sequence to shorten the repetition time for the quantification of the proton density of bone marrow in calcaneus.

### DETAILED DESCRIPTION OF THE INVENTION

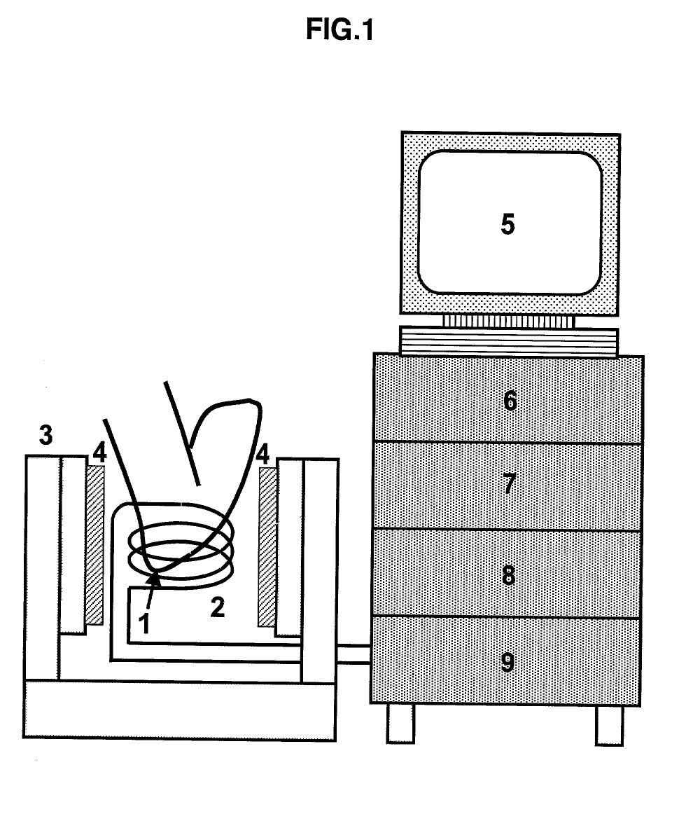

FIG. 1 schematically illustrates an MRI system with a permanent magnet, gradient coil, RF probe, and MRI console for imaging of a heel, as an exemplary embodiment of this invention.

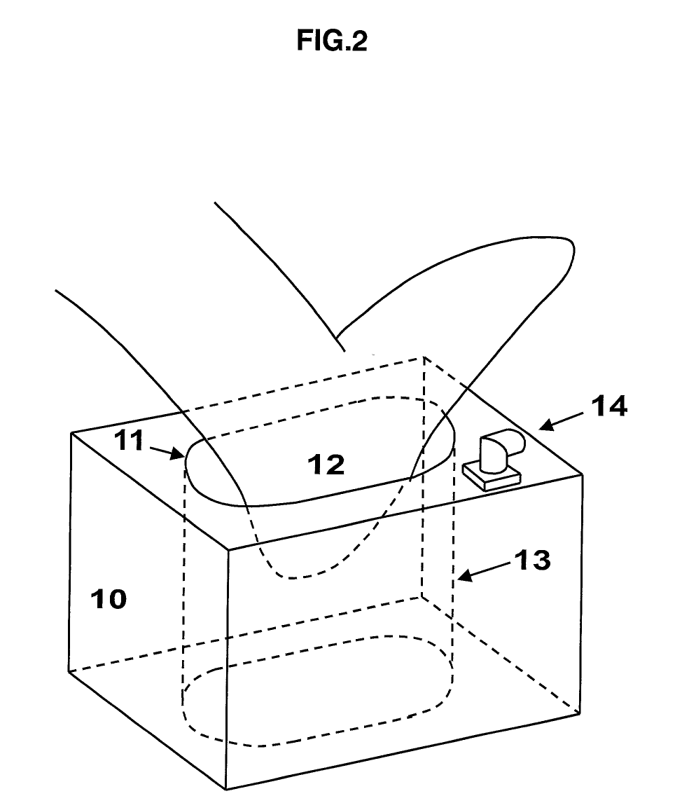

In FIG. 1, the heel 1 is inserted into the RF coil 2 placed between the permanent magnet 3 and gradient coil 4. The permanent magnet produces a homogeneous magnetic field of about 0.21T over the 12 cm diameter spherical volume in the central region of the magnet gap, of which width is 16 cm. The volume of the homogeneous magnetic field is sufficient to image a heel and external reference phantoms at the same time. The RF coil is actually stored in an RF shielded box as shown in FIG. 2 to eliminate the electromagnetic coupling between the RF coil 2 and gradient coil 4.

MRI measurements are controlled by the MRI console, which consists of a display monitor 5, computer system 6, RF transceiver 7, gradient driver 8, and RF transmitter 9. The computer system includes a digital signal processor (DSP) board for the MRI pulse programmer, a direct digital synthesizer (DDS) board for generation of a radio frequency source for the NMR transceiver, and an analog-to-digital converter (ADC) board for digitization of the NMR signal.

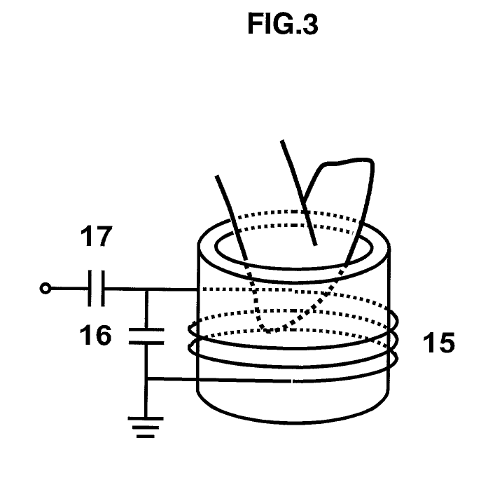

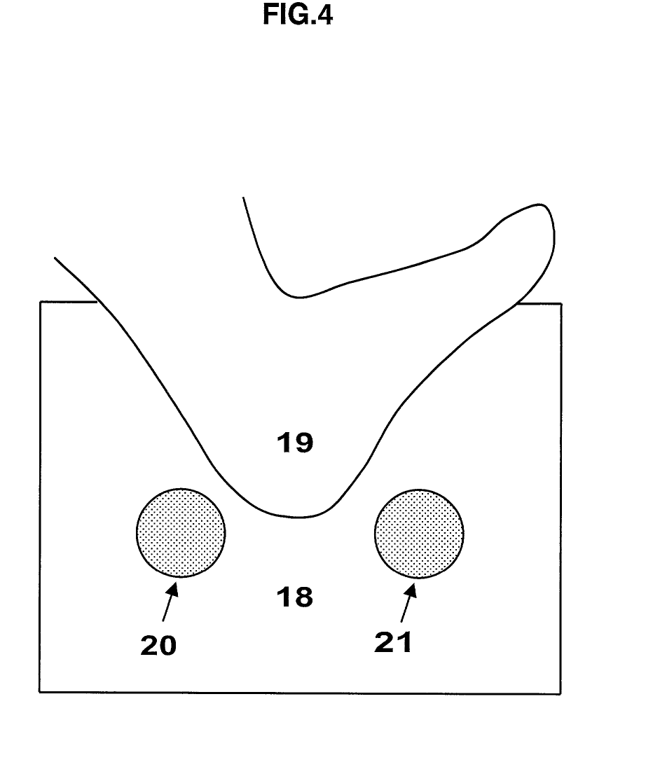

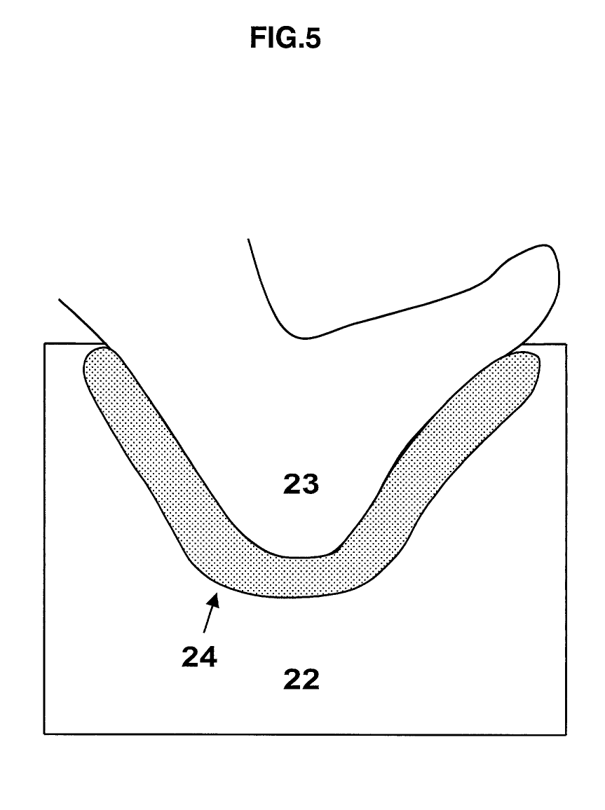

The RF transceiver 7 is used to generate RF pulses for excitation of the proton spins of the heel and convert the NMR signal modulated at the Larmor frequency at around 8.9 MHz to a lower frequency (several tens of kHz) having spatial information of the proton spins. The gradient driver 8 is used to supply electric currents to the gradient coil 4 according to waveforms generated by the pulse programmer. The transmitter 9 is used to supply RF currents to the RF coil 2. FIG. 2 schematically illustrate the RF probe used in the system. The RF probe box 10 is made of brass plates with an oval aperture 11 in the upper side. The dimension of the box is 22 cm (width)×16 cm (height)×13 cm (depth) and that of the aperture is 16 cm (long axis)×8 cm (short axis). The aperture can accommodate heels of most female adult patients as depicted as 12. The probe has an open-bore RF coil support 13 made of acrylic plates, which goes from the upper aperture down to another aperture in the lower side of the probe box. A solenoid coil with an oval cross-section is wound over the coil support. In the open-bore coil support, a support pad for a heel and external reference phantoms is inserted as will be depicted in FIG. 4 and FIG. 5. An RF connector 14 is attached on the upper side of the probe box to supply RF currents for excitation of proton spins and to receive NMR signals from proton spins. An LC tank circuit is schematically depicted in FIG. 3. In this figure, although the RF coil 15 is depicted as a solenoid with a circular cross-section, the solenoid coil with the oval cross-section which is wound on the RF coil support 13 can be also used for the tank circuit. The capacitor 16 is used mainly for tuning of the tank circuit to the resonance frequency (8.9 MHz) and the capacitor 17 is used mainly for impedance matching to 50 ohms.

FIG. 4 is a schematic cross-sectional depiction of a support pad 18 for a heel 19 and external reference materials denoted by 20 and 21, inserted in the open-bore of the RF probe shown in FIG. 2. For quantification of proton densities of bone marrow in calcaneus, to fix the heel and to acquire MRI images together with known reference materials such as water is essential, because tuning of the tank circuit varies person to person dependent on the size and position of the foot. In this figure, polystyrene foam is used for the support pad 18 and CuSO₄ water solution in cylindrical polyethylene bottles is used as the external reference material.

FIG. 5 shows an alternative embodiment for the support pad 22. A heel 23 is directly placed on the external reference material 24 which is flexible to fit any shape of a foot to improve the precision of RF field inhomogeneity correction.

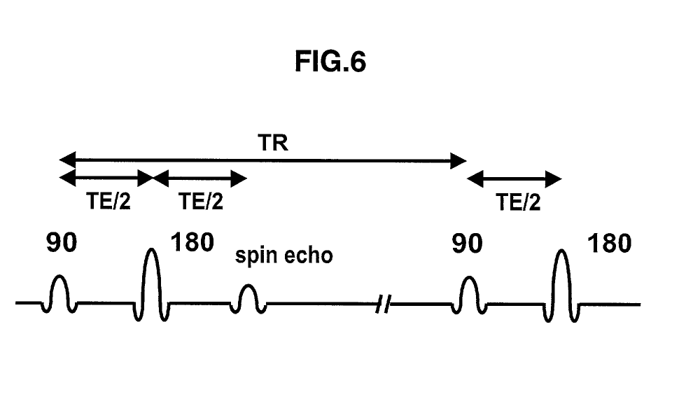

FIG. 6 is a standard 2D spin-echo pulse sequence used to quantify the proton density of bone marrow in calcaneus. Image intensity I(x, y) acquired with the spin-echo sequence can be expressed except the J modulation effect of lipid as follows:

I(x,y)=kf(x,y)ρ(x,y){1−p(x,y)exp(−TR*/T₁(x,y))}exp(−TE/T₂(x,y)), (1)

where k is a constant, f(x,y) represents spatial variation of image intensity for a uniform sample, which is determined by inhomogeneity of static magnetic field, magnetic field gradients, RF magnetic field, and coil sensitivity (in principle the same as distribution of RF magnetic field), p(x,y) represents a factor reflecting longitudinal magnetization, which is unity for perfect 90 degree excitation over the cross-sectional plane, TR* is a time close to the repetition time TR, T₁(x,y) and T₂(x,y) are T₁ and T₂ distributions in the plane.

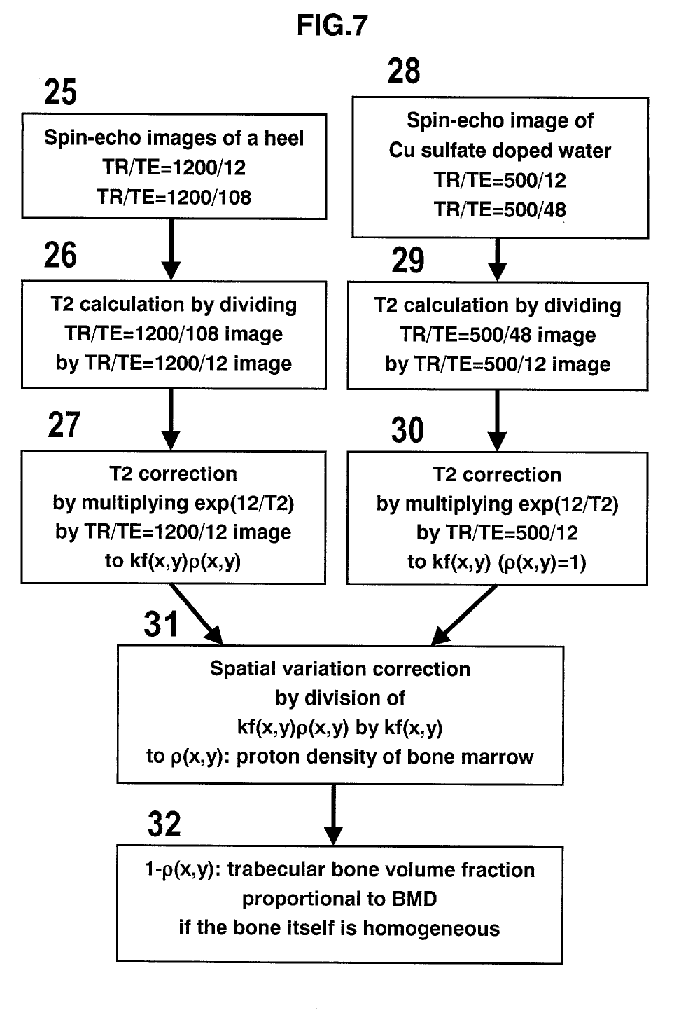

FIG. 7 shows a flow diagram of one method to quantify the proton density of bone marrow in calcaneus. To quantify the proton density ρ(x,y), spin-echo sequences with TR/TE=1200 ms/12 ms and TR/TE=1200 ms/108 ms are used for the heel measurements as denoted by 25. Since the T₁ of the protons of the bone marrow in calaneus is about 250 ms at 8.9 MHz, MR image intensities at calcaneus acquired with the above sequences can be expressed as

I₁(x,y)=kf(x,y)ρ(x,y)exp(−12/T₂(x,y)) (2)

I₂(x,y)=kf(x,y)ρ(x,y)exp(−108/T₂(x,y)). (3)

By using above two equations, T₂ (x, y) and kf(x, y)ρ(x,y) are calculated as denoted by 26 and 27. For the measurement of kf(x,y), a CuSO₄ water solution phantom of which T₂ is about 43 ms is placed in the RF coil instead of a heel and imaged with the same spin-echo sequences with TR/TE=500 ms/12 ms and TR/TE=500 ms/48 ms as denoted by 28. By using these images, kf(x,y) is calculated when the proton density of water is defined as unity as denoted by 29 and 30. The proton density ρ(x,y) of the bone marrow when the proton density of water is defined as unity is thus calculated by dividing k(x,y)p(x,y) by f(x,y) as denoted by 31. The trabecular bone volume fraction is then computed as 1−ρ(x,y), as denoted by 32.

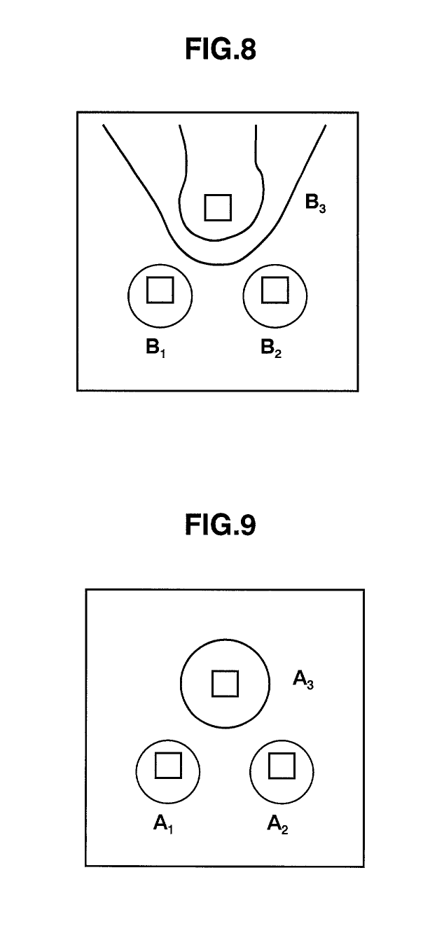

FIG. 8 and FIG. 9 schematically show cross-sectional images of a heel and a water phantom with external reference phantoms. For actual calculations for proton densities and T₂ relaxation times, mean values in the squares depicted in FIG. 8 and FIG. 9 are used because random noise is superimposed on the MR images and the precision is limited.

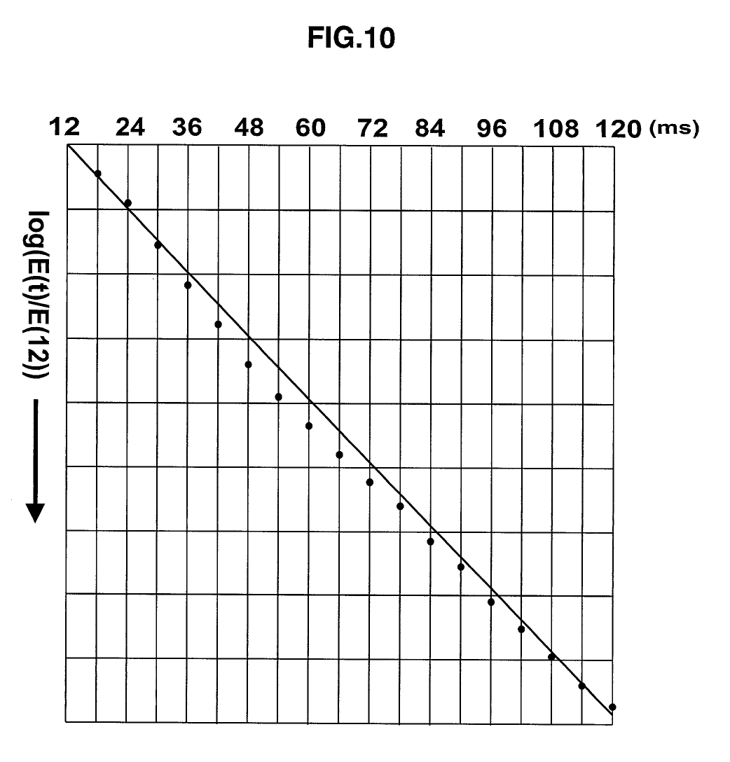

FIG. 10 is a semilogarithmic plot of spin-echo image intensities of a plant oil phantom measured against the echo time, showing the J modulation effect on the spin-echo intensities. The similar property is observed for the bone marrow in calcaneus because the bone marrow has a similar chemical composition as the plant oil. The J modulation effect, which is seen as the deviation of the spin-echo signal shown from the straight line, is shown as an oscillation with about 100 ms period. Thus the T₂ decay of the lipid protons can be corrected reasonably using the images acquired with TE=12 ms and TE=108 ms.

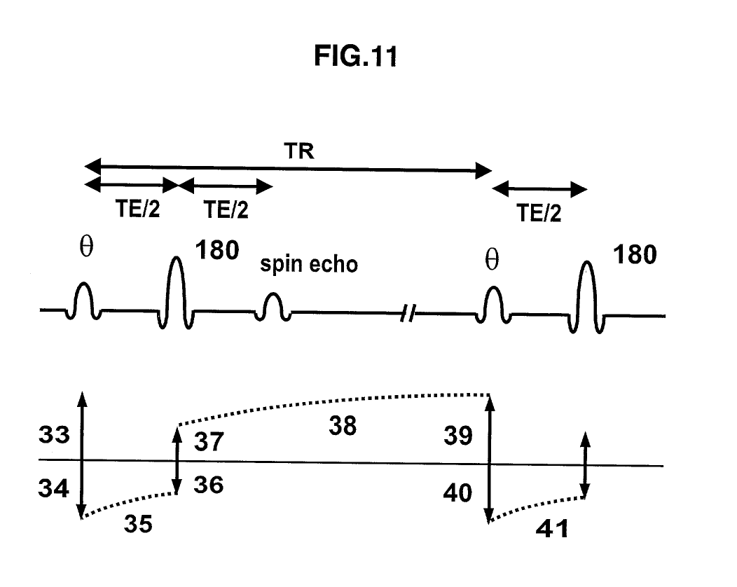

FIG. 11 is a large flip-angle excitation 2D spin-echo pulse sequence to shorten the repetition time for the quantification of the proton density of bone marrow in calcaneus. By setting the excitation flip angle to an angle between 90 and 180 degree, the recover of the longitudinal magnetization becomes faster as depicted in FIG. 11. The arrows denoted by 33 to 41 show a time variation of longitudinal nuclear magnetization.

While only a few specific exemplary embodiments of this invention have been described in detail, those skilled in the art will readily appreciate that many variations and modifications may be made in these exemplary embodiments while yet retaining many of the novel features and advantages of this invention. Accordingly, all such modifications and variations are intended to be included within the scope of the appended claims.

What is claimed is:

1. An improved MRI system having a compact magnet and an RF coil which accommodate a human extremity such as a heel and external reference materials to measure the proton density of bone marrow to quantify trabecular bone volume fraction.

2. An improved MRI system as in claim 1 wherein said RF coil is stored in an electromagnetically shielded box to eliminate the electromagnetic coupling between the RF coil and gradient coil.

3. An improved MRI system as in claim 2 wherein said RF probe box has an oval aperture to accommodate a human extremity such as a heel.

4. An improved MRI system as in claim 2 wherein a support pad for a heel and external reference materials is inserted into said RF probe open-bore to fix the heel and to acquire calibration image data at the same time.

5. An improved MRI system as in claim 4 wherein said external reference material is flexible and can be used to fix a heel and to improve the precision of measurement of the RF magnetic field.

6. An improved MRI system as in claim 1 wherein large flip angle spin-echo imaging sequences are used to shorten the repetition time of the sequences.

7. A method for quantifying proton densities in inhomogeneous static magnetic field, magnetic field gradients, and RF magnetic field, comprising the step of:
(a) measuring cross-sectional MR images of a human extremity such as a heel together with external reference materials with two spin-echo sequences in which the repetition time TR is long enough for signal saturation for the proton spins in the region of interest and one spin-echo time is set as short as possible and the other spin-echo time is set longer than that of the shorter one by about 100 ms to eliminate the J modulation effect on T₂ decay correction;
(b) dividing the image intensity of said MR image with the longer spin-echo time by that of said MR image with the shorter spin-echo time to calculate T₂ relaxation time and spin density distribution including the spatial variation function for the imaging area;
(c) correcting the T₂ decay by multiplying exponential function of the shorter spin-echo time divided by T₂, by said MR image with the shorter spin-echo time;
(d) measuring cross-sectional MR images of a reference material such as paramagnetic salt doped water at the position where the human extremity was placed together with said external reference materials with two spin-echo sequences in which the repetition time TR is long enough for signal saturation for the proton spins and one spin-echo time is set as short as possible and the other spin-echo time is set longer than that of the shorter one by about T₂ relaxation time of the protons of the reference material to optimize the T₂ decay correction;
(e) dividing the image intensity of said MR image of the reference material with the longer spin-echo time by that of said MR image of the reference material with the shorter spin-echo time to calculate T₂ relaxation time and the spatial variation function for imaging area;
(f) correcting the T₂ decay by multiplying exponential function of the shorter spin-echo time divided by T₂, by said MR image of the reference material with the shorter spin-echo time; and
(g) dividing the T₂ corrected MR image calculated in (c) by the T₂ corrected MR image calculated in (f) to obtain the proton density of the human extremity such as a heel when the proton density of the reference material is defined as unity.

8. The method of claim 7, wherein the image intensity calculations are performed using means over some regions of interests to improve the data fluctuation.
